(19) United States
(12) Patent Application Publication
(10) Pub. No.: US 2019/0224654 A1
(43) Pub. Date: Jul. 25, 2019

(54) OLIGOMERISATION CATALYST

(71) Applicant: The Petroleum Oil and Gas Corporation of South Africa (PTY) Ltd, Cape Town (ZA)

(72) Inventors: Masikana Millan Mdleleni, Cape Town (ZA); Yusaf Isa Makarfi, Cape Town (ZA); Nonyameko Prudence Sincadu, Cape Town (ZA); David Key, Cape Town (ZA)

(21) Appl. No.: 15/562,370

(22) PCT Filed: Jan. 29, 2016
(86) PCT No.: PCT/ZA2016/050005

§ 371 Date: Sep. 27, 2017

(30) Foreign Application Priority Data
Jan. 29, 2015 (ZA) 2015/00657

(51) Int. Cl. B01J 29/46; B01J 37/02; B01J 37/04; B01J 37/00; B01J 29/40; B01J 37/08; B01J 35/02; C01B 39/40; C07C 2/12

(52) U.S. Cl. CPC B01J 29/46; C01P 2006/14; B01J 37/04; B01J 37/0201; B01J 37/0018; B01J 29/405; B01J 37/08; B01J 35/026; C01B 39/40; C07C 2/12; B01J 2229/186; C01P 2002/70; C01P 2002/82; C01P 2004/03; C01P 2006/12; B01J 37/0236

(57) ABSTRACT

The invention provides a method of manufacture of a catalyst, the catalyst as such and its use in olefin oligomerisationand. The method for making an oligomerisation catalyst includes the steps of mixing alumina, silica, metal oxide, a template source; and effecting the deactivation of the Bronsted sites by masking them with metal ions, preferably either (i) during synthesis by directly adding the metal as ion in a one-pot or direct synthesis, or (ii) after intermidiate conversion to the H+-form by in turn either (ii-a) impregnating the latter with a metal salt, or (ii-b) thy-mixing a metal salt with it.

### TECHNICAL FIELD

This invention relates to a method for making a catalyst, a catalyst and a process using the catalyst.

### BACKGROUND TO THE INVENTION

The inventor is aware of the need for catalysts for the oligomerisation of olefins for use in processes for conversion of olefins to diesel (COD), which catalyst have a high conversion rate and good selectivity. It is also not desirable for these catalysts to crack hydrocarbons.

It is an object of the invention to provide olefin oligomerisation catalysts with high conversion rate, good selectivity and which cause no perceivable cracking reactions.

### GENERAL DESCRIPTION OF THE INVENTION

According to the invention there is provided a method for making an oligomerisation catalyst, which method includes the steps of:
mixing alumina, silica, metal oxide, a template source; and
effecting the deactivation of the Bronsted sites by attachment of the metal thereto.

In one embodiment, co-crystallisation, of the invention aluminosilicate starting gel is prepared from silica gel, aluminum nitrate nona hydrate and aluminium sulphate. 1 M nitric acid is used to control the pH of the gel. Alkyl salts are used as templating agents and one of nitrate of zinc, iron and nickel (M) were added to the mixture for co-crystallisation. The SiO₂/MO ratio can be selected between 100 and 10. Co-crystallisation can be carried out in a hydrothermal synthesis reactor, preferably with a Teflon cup. The hydrothermal synthesis is carried out for about 24 hours at a temperature of about 190° C. After crystallisation the reactor is allowed to cool followed by filtration with distilled water until the filtrate had the same pH as the distilled water. The obtained crystals are then dried and calcined. The crystals were then protonated by stirring with 1 M ammonium chloride at 90° C. The crystals are then again filtered with distilled water until the pH of the filtrate is the same as the distilled water. The crystals were the again dried and calcined.

In another embodiment, incipient wetness impregnation, a protonised form of zeolites can be obtained by calcining NH₄-ZSM-5 (zeolyst pellets) in a stream of Nitrogen at about 500° C. for about 3 hours. Nitrates of zinc, iron and nickel, M, were dissolved in water is then used to achieve a loading of 1 to 10% via incipient wetness. The impregnated catalyst is then dried and calcined.

In another embodiment, mechanical mixing, the protonised form of NH₄-ZSM-5 is mechanically mixed with nitrates of M, M being selected from zinc, iron and nickel. The mixture is then calcined with MO % being 1, 0.2 and 0.04.

The invention also extends to a method for oligomerising olefins, which method include the step of contacting olefins with the catalysts as described above under oligomerisation reaction conditions.

The method also extends to a catalyst described herein and as manufactured by the method as described above.

### DETAILED DESCRIPTION OF THE INVENTION

The invention is now described by way of example with reference to the accompanying graphs and figures.

Catalyst Synthesis

Co-crystallisation

Zeolite materials were synthesized hydrothermally from aluminosilicate gel; water glass supplied by Kimix was used as a source of silica gel. Aluminum nitrate nona hydrate and aluminium sulphate were used as the Alumina sources. 1 M nitric acid was used to control the target pH of the gel. Different alkyl salts were used as templating agents. Nitrates of zinc, iron and nickel were incorporated into the batch mixtures for co crystallization, the SiO₂/MO was varied to be 100, 20 and 10. Nine samples were produced using this technique.

TABLE 2.1
List of co crystallized zeolites catalysts from template,
1,6hexamethylenediamine (HMD)

| S/No. | Catalyst | SiO₂/MO |
|---|---|---|
| 1 | HMD(Fe1) | 100 |
| 2 | HMD(Ni1) | 100 |
| 3 | HMD(Zn1) | 100 |
| 4 | HMD(Fe2) | 20 |
| 5 | HMD(Ni2) | 20 |
| 6 | HMD(Zn2) | 20 |
| 7 | HMD(Fe3) | 10 |
| 8 | HMD(Ni3) | 10 |
| 9 | HMD(Zn3) | 10 |

A starting gel was prepared from the alumina, silica, metal oxide and template sources. The crystallization of the obtained gel mixture was carried out in stainless steel hydrothermal synthesis reactor with a teflon cup for a period of 24 hours at a temperature of 190° C. After the crystallization, the autoclave was allowed to cool before the solid phase was separated from the liquid by filtration. Filtration was achieved using distilled water until the filtrate had the same pH as the distilled water. The crystals obtained were allowed to air dry and later oven dry at temperatures between ambient to 120° C. after which they were calcined in a stream of air for 6 hours using a modified muffle furnace.

To obtain the H form of the zeolites, the crystals were protonated using 1 M ammonium chloride solution while stirring at a temperature of 90° C. The solution was filtered using distilled water until no difference was observed between the pH of the distilled water and that of the filtrate.

The obtained crystals were then oven dried over night at temperatures of 120° C. before calcination at 450° C. for 3 hours.

Incipient Wetness Impregnation

NH4-Zsm-5 (zeolyst pellets) obtained from zeolyst was calcined in a stream of Nitrogen at 500° c. for 3 hours to obtain the protonised form of the zeolites. Nitrates of Zn, Fe and Ni were dissolved in distilled water and used to achieve a loading of 1%,3%,7% and 10% via incipient wetness impregnation. The impregnated catalyst was allowed to oven dry overnight at a temperature of 120° c., after which the samples were calcined in a stream of air at 450° C. for 3 hours. Using this method, 15 different samples were produced.

TABLE 2.2
List of catalysts synthesized by incipient wetness impregnation

| | | Metal Oxide loading |
|---|---|---|
| S/No. | Catalyst | (%) |
| 1 | 1% FeOZSM-5 | 1 |
| 2 | 1% NiOZSM-5 | 1 |
| 3 | 1% ZnOZSM-5 | 1 |
| 4 | 3% FeOZSM-5 | 3 |
| 5 | 3% NiOZSM-5 | 3 |
| 6 | 3% ZnOZSM-5 | 3 |
| 7 | 5% FeOZSM-5 | 5 |
| 8 | 5% NiOZSM-5 | 5 |
| 9 | 5% ZnOZSM-5 | 5 |
| 10 | 7% FeOZSM-5 | 7 |
| 11 | 7% NiOZSM-5 | 7 |
| 12 | 7% ZnOZSM-5 | 7 |
| 13 | 10% FeOZSM-5 | 10 |
| 14 | 10% NiOZSM-5 | 10 |
| 15 | 10% ZnOZSM-5 | 10 |

2.1.3. Mechanical Mixtures

Protonised form of the NH4-ZSM-5 zeolites obtained from zeolyst was mechanically mixed with nitrates of iron, nickel and zinc. The obtained catalytic systems were calcined in a stream of air at 450° C. for 3 hours, using a MO %(wt) of 1, 0.2 and 0.04. Using this technique, the following samples in table 2.3 were obtained.

TABLE 2.3
List of catalysts synthesized as mechanical mixtures

| S/No. | Catalyst | % MO in mixture |
|---|---|---|
| 1 | MMFe1 | 0.04 |
| 2 | MMFe2 | 0.2 |
| 3 | MMFe3 | 1 |
| 4 | MMNi1 | 0.04 |
| 5 | MMNi2 | 0.2 |
| 6 | MMNi3 | 1 |
| 7 | MMZn1 | 0.04 |
| 8 | MMZn2 | 0.2 |
| 9 | MMZn3 | 1 |

Hexene conversion was used to determine the activity of the catalysts. It will be appreciated that the catalyst are not limited to oligomerisation of hexene. The impregnation of Fe, Zn and Ni oxides on commercially obtained ZSM-5 catalyst showed a remarkable change in the selectivity towards C10+ hydrocarbons. At 1% ZnO loading, selectivity to C10+ hydrocarbons was seen to be better than that of the commercially obtained catalyst (FIG. 8). Though 1% loading of FeO showed similar selectivity to C₁₀₊ hydrocarbons as the commercially obtained ZSM-5 catalyst it was not as high as that obtained over the system promoted with 1% ZnO. However, NiO promoted catalyst showed better selectivity towards C₆-C₉ hydrocarbons, and it appears that at 1% NiO loading, the catalytic system does not favor the formation C10+ as final products. However it should be noted that the conversion was well over 86% and based on this, 1% NiO could have a very good inter-oligomerisation/oligomerisation activity and a potential system for production of high octane gasoline from products of primary distillation of crude oil.

In FIG. 9, it is seen that increasing the FeO loading via IWI from 1 through 5 to 10% results in catalyst selectivity towards C10+ passing through a maximum. At 5% loading, the selectivity for C10+ in the liquid product was 72% while at 1% and 10% loadings, the selectivities were 40% and 46% respectively. The mirror image of the selectivity to C₆-C₉ hydrocarbons suggests a possible relationship between the reactions consuming the middle fraction and those producing the heavier fractions. To determine the optimal FeO loading, a 7% FeoZSM-5 was synthesized. This however further confirmed that within the series, 5% loading favored the highest formation of C10+ hydrocarbons.

Table 3.2 shows the individual concentration of compounds as obtained from the liquid GC analysis. The formation of C₁₀₊ hydrocarbons that was observed to be highest over 5% FeOZSM-5 is seen to have considerable amounts of C14-C17+, suggesting that the cracking of longer chain hydrocarbons was more favourable than in the other catalytic systems. It therefore seems preferable to develop catalytic systems that will have limited cracking activities to aid in realising the goals of the COD process.

TABLE 3.2
Product distribution of hexene conversion over Zeolite catalysts
T = 350° C., WHSV = 2 h⁻¹

| | 1% | 5% | | |
|---|---|---|---|---|
| COMPOUNDS | FeOZSM5 | FeOZSM-5 | % 10FeOZSM-5 | ZSM-5 |
| C1-5 | 13.77 | 3.17 | 19.30 | 27.63 |
| C6 | 4.98 | 1.53 | 4.39 | 1.02 |
| C7 | 1.91 | 1.69 | 1.38 | 2.03 |
| C8 | 14.66 | 10.49 | 7.43 | 11.08 |
| C9 | 24.36 | 10.83 | 17.29 | 16.82 |
| C10 | 17.94 | 16.49 | 13.44 | 9.16 |
| C11 | 9.7 | 11.93 | 6.77 | 5.32 |
| C12 | 0.14 | 10.24 | 4.57 | 3.74 |
| C13 | 2.31 | 2.04 | 9.34 | 9.00 |
| C14 | — | 13.46 | 11.97 | 11.49 |
| C15 | 10.22 | 9.54 | 3.83 | 2.34 |
| C16 | — | 4.57 | 0.29 | 0.37 |
| C17+ | — | 3.33 | — | — |

Loading ZnO at 1% did not result in any significant change in selectivities to C₁₀₊ when compared to the performance of the commercially obtained catalyst.

The 3% ZnZSM-5 showed the best selectivity to C10+(58%) hydrocarbons over the Zn IWI series (FIG. 10). The hexene conversion was very good over all the ZnO catalysts promoted by IWI. This catalyst series (which one) (this refers to the system immediately discussed and is acceptable to write in this way since there is only one series mentioned before the statement)though not looking promising for the COD process could be applicable in other fuel synthesis reactions.

A further catalyst series for the COD process is the NIO ZSM-5 system, as shown in FIG. 11, this catalyst series continued to have better selectivity to the heavier hydrocarbons as the loading was increased from 1 to 10%. It is important to note that throughout the series, the hexene conversion was more than 91% and a direct relationship exists between 5-10% loading and selectivity for C₁₀₊.

The presence of phases other than those of ZSM-5 systems in the PXRD patterns of the co-crystallized samples with SiO2/MO=10 made it interesting to study the activities of same catalysts in hexene conversion. All the three catalysts as shown in FIG. 14 performed worse than ZSM-5 catalyst in both conversion and selectivity towards C₁₀₊hydrocarbons. However it will be interesting to further study the 20XII3JKHMD(Ni-3) system as it showed a relatively good conversion of hexene. The Fe and Zn co crystallized systems had conversions of 12 and 1.55 respectively. This poor conversion activity is possibly the result of silicates formed due to the high MO incorporated resulting in non-formation of the desired ZSM-5 catalysts.

Table 3.3 suggests that during co-crystallisation with low loadings of SiO₂/MO=100, there was a better formation of the zeolite frame work. At very low zinc oxide co crystallized sample, the catalyst obtained 20XII31KHMDZn1 exhibits potential for application in the COD process, There is therefore a need to co crystallize with the right amount of Metal oxides in order to achieve a good conversion.

TABLE 3.3
Performance of SiO₂/MO = 100 systems in hexene conversion

| Para- | | | |
|---|---|---|---|
| meter | 20XII31KHMDFe1 | 20XII25JHMDNi1 | 20XII31KHMDZn1 |
| Con- | 66.89 | 79.25 | 89.75 |
| version | | | |
| XC1-5 | 24.43 | 19.51 | 15.09 |
| XC6-9 | 62.78 | 47.71 | 71.3 |
| XC10+ | 12.79 | 32.78 | 13.61 |

From FIG. 13, it can be seen that the highest conversion was achieved at SiO2/NiO of 100 and this declined as the ratio increased further confirming that at certain loadings, formation of the target ZSM-5 framework is hindered by the presence of other metal compounds that do not form part of the zeolite framework. The same trend is observed for selectivity towards C₁₀₊, however the relationship between the conversion and selectivity to C10₊ is proportional but not linear.

A study of mechanical mixtures can give an insight to the possibility of using binary catalyst systems or multi bed reactors. Interestingly enough all the catalysts synthesized as mechanical mixtures showed very good conversion of hexene with a relatively high selectivity towards C10+. As can be seen from table 3.4, MMFe1 showed the highest selectivity towards C10+ hydrocarbons while MMFe3 had the lowest selectivity towards C10+. Generally from table 3.4 it is evident that mechanical mixtures could also serve as potential COD catalysts.

TABLE 3.4
Hexene conversion and product distribution over mechanical mixtures.

| | | Selectivity | | |
|---|---|---|---|---|
| Catalyst | Conversion | C1-C5 | C6-C9 | C10+ |
| MMFe1 | 97.91 | 1.20 | 18.55 | 80.25 |
| MMFe2 | 97.29 | 1.79 | 25.82 | 72.39 |
| MMFe3 | 95.75 | 1.70 | 46.18 | 52.13 |
| MMZn1 | 97.26 | 1.52 | 33.84 | 64.64 |
| MMZn2 | 98.54 | 1.74 | 35.34 | 62.92 |
| MMZn3 | 99.55 | 1.13 | 27.98 | 70.89 |
| MMNi1 | 98.18 | 0.2 | 26.35 | 73.45 |
| MMNi2 | 97.93 | 2.65 | 32.58 | 64.77 |
| MMNi3 | 96.48 | 2.26 | 21.18 | 76.56 |

Catalyst Characterization

The synthesized catalysts were characterised using different techniques including XRD, FTIR, SEM, EDS, BET and TPD, A Perkin Elmer Spectrum 100 FTIR Spectrometer was used for the infra-red spectroscopic studies. The X-ray diffractometry was achieved using a BRUKER AXS D8 Advance (Cu-Kα radiation λKα₁=1.5406 Å) 40 kV. The Hitachi X-650 Scanning Electron Microscope (Tungsten filament, EHT 20.00 kV) and LEO 1450 Scanning Electron Microscope (Tungsten filament, EHT 20.00 kV) were used for the SEM imaging and EDS analyses. BET studies were conducted on a Micromeritics tristar 3000. NH₃ TPD was achieved on an AUTOCHEM 2910.

The catalyst activity was tested using a glass fixed bed reactor with hexene as feed. A glass reactor with a frit and glass wool were used to mimic a fixed bed reactor. Using a syringe pump, hexene was fed into the reactor at a flow rate determined by the mass of the catalyst so as to attain the required WHSV. The products were cooled through a condenser before the liquid was collected in a round bottom flask and the gases as overheads were connected to a calibrated bubble meter so as to measure the flow rate of the gas produced. The products were analysed using a varian 3400 GC with a petrocol 100 m×0.25 m column and an FID.

Powder X Ray Diffraction

The Co-crystallisation of ZSM-5 with metal oxides showed significant changes in phase composition as the SiO2/MO ratio was varied. It is seen from figures A-1,A-2 and A-3 that though all the samples showed presence of ZSM-5 important 2θ peaks, the sample was purer when the SiO2/MO was lowest. The characteristic peaks at 2θ=7.92 and 8.84 representing (011) and (020) planes of crystal structures are present in all samples but least intense in the 20XII03JHMD(Fe3). The presence of the 26°(2θ) peak corresponding to quartz is seen in many samples, this suggests the presence on un utilized SiO₂ in the zeolite frame network. Interestingly enough, this peak is more prominent in the samples with the lowest SiO2/MO. Nonetheless at these loading the PXRD patterns show good crystallinity

A close look at figure A-1 shows that at SiO₂/ZnO=20 there is a significant amount of amorphous phase; explain the graphs as the writing or numbering on the graphs is very small. While 20XII03JHMD(Zn1) and 20XII03JHMD(Zn3) showed better crystallinity, it is worth noting the intensity of the peak at is 37°(2θ) in 20XII03JHMD(Zn3) that is not so intense in a commercially obtained ZSM-5 pattern. Figure A-2 shows a decrease in the crystallinity as the composition of NiO increases in the co crystallized system. It is seen that while 20XII03JHMD(Ni2) is closest in pattern to the ZSM-5, it is not as crystalline as 20XII03JHMD(Ni1), which however has the quartz peak indicating the non-purity of that sample. While the trend in figure A-3 is similar to that in A-2, hematite phase is detectable in the 20XII03JHMD (Fe3) pattern. We suggest that non detection of significant oxide phases is as a result of their relative low concentrations in the samples

Figure A-4, A-5 and A-6 show the patterns of ZSM-5 catalysts that were obtained from mechanical mixtures ZSM-5 and metal oxides. All systems synthesized as mechanical mixtures showed good crystallinity and purity. No phases showing the presence of were visible, suggesting the aimed low loadings and/ or well incorporation into the ZSM-5 network. All the catalysts had characteristic peaks at 2θ=7.92, 8.84, 23.12 and 23.8° corresponding to (011), (020), (051) and (033) planes.

Now looking at the IWI, MO loadings of up to 10% did not show any detrimental changes to the purity or phase composition of 15 samples synthesized with nitrates of iron. zinc and nickel as precursors of MO (FIG. 3). Though the presence of some of the MO is slightly detected, their phases are so minute showing no significant that effect on neither the purity nor the phase composition of the synthesized samples.

Fourier Transform Infrared Spectroscopy

FIG. 1 shows the FTIR spectra of ZSM-5 catalysts impregnated with metal oxides. It is seen on FIG. 1A that, the characteristic band of the pentasil structure at 543 cm⁻¹ is present in all the synthesized samples; however the intensity is seen to be least for the NiO impregnated catalyst. The internal asymmetric stretch band near 1080 cm⁻¹is present in all the samples. The Si-O-T band around 800 cm⁻¹ is present but not very intense in all the three samples. The observed trend in the FTIR spectra at other loadings is very similar to that at 1% loading. The FTIR spectra of Co crystallized samples are seen to be very different from those obtained for the samples prepared by IWI. FIG. 2A shows low intensity peaks at 543 cm⁻¹ which are attributed to the double ring vibration of the pentasil structure. The T-O-T peaks at 450 cm⁻¹are present in all the spectra, the very low intensity of the double ring vibration peaks suggests very low crystallinity of these samples, this we suggest has to do with the formation of silicates that are not fully incorporated into the ZSM-5 network structure. Interestingly enough the 800 cm⁻¹ peak attributed to Si-O-Me band is more pronounced here than in the samples prepared by IWI. The bands attributed to the internal asymmetric stretch are also well pronounced, however the 1280 cm⁻¹ band is not well identified

FIG. 2B, shows a drastic decrease in the intensities of the 1080 cm⁻¹band for the 20XII03JHMD(Fe3) and 20XII03JHMD(Zn3) samples, however this drastic change is not observed in the sample.

co crystallized with Ni nitrate at the same SiO₂/MO. The same trend is observed for the peaks at 450 cm⁻¹. For the mechanical mixtures synthesized with very low MO:ZSM-5, all characteristic bands for ZSM-5 were seen to be present however some peaks around the 2000 cm⁻¹ (FIG. 2A) not common to ZSM-5 catalysts are observed in MMNi1, they are also observed to begin emerging in MMFe1. On FIG. 3, it is seen that an increase in MO:ZSM-5 leads to the peaks around 2000 cm⁻¹ being more pronounced for MMFe3 and MMZn3. It is worth noting the MMNi disagree with the two other samples inversely with regards to the peaks around 2000 cm⁻¹.

Morphology

The SEM images of co crystallized ZSM-5 and metal oxides with SiO₂/MO=99 are shown in FIG. 3-5.

From FIG. 3, It is observed that the formation of prism like crystals has begun, however at this stage there is still a lot of material in the sample that is yet to take a definite shape.

The micrograph on FIG. 4 shows the presence of a well-defined prism formed. However just like in the case of SiO2/FeO=99, there is no defined shape that could be attributed to the particles

For the SiO₂/ZnO=99 systems, no particular crystal shapes are seen.

By probing inner on the co-crystallised ZnO system, well defined cylindrical shaped crystals are seen (FIG. 4A). The cylindrical particles are about 2.5 μm in size. The EDAX of 20XII03JHMD (Zn3) shows the presence of Zinc in a ratio of 5 to 1 silicon. The high amount of carbon we attribute to the coating of the samples during preparation. From the micrographs and EDAX, it is evident that for both Feo and ZnO systems, there is a formation of well-defined crystal structures, the non-formation of these forms throughout the sample could be as a result of the high loading distorting the formation of ZSM-5 framework.

3.4. Ammonia Temperature-programmed Desorption

The acidity of the synthesized samples was determined using NH₃-TPD. The samples in FIG. 7 all show the presence of acid peaks corresponding to the TCD signal for the desorbed ammonia. The first peaks are attributed to the Physisorbed ammonia. Subsequent peaks at about 400° C. and 600° C. correspond to those of medium and strong acidic sites. The chemisorbed peaks at lower temperatures correspond to the Bronsteid sites while the peaks at higher temperatures correspond to the Lewis sites. The 3% FeZSM-5 has all three peaks present. It should be noted that for safety reasons, the TPD was run up to 600° C. hence only the beginning of the strong acid sites was observed. The Co- precipitated samples seem to show larger peaks corresponding to the strong sites and very tiny peaks corresponding to the medium strength sites, indicating the presence of more Lewis sites which can be attributed to the framework distortion by the substitution of some of the Al by Me resulting in a change in electron density.

### BET

TABLE 3.1
BET Analyses of ZSM-5 catalytic systems promoted by IWI

| | Surface area | Total pore | |
|---|---|---|---|
| Catalyst | (m²/g) | volume (cm³/g) | Pore size (Å) |
| 1% FeOZSM-5 | 268.2119 | 0.279021 | 47.4765 |
| 1% NiOZSM-5 | 259.4908 | 0.293095 | 47.63 |
| 1% ZnOZSM-5 | 263.9722 | 0.29429 | 49.0346 |
| 3% NiOZSM-5 | 229.2479 | 0.263936 | 48.919 |
| 3% FeOZSM-5 | 268.4445 | 0.261027 | 45.0361 |
| 3% ZnOZSM-5 | 305.9381 | 0.319057 | 45.7013 |
| 5% FeOZSM-5 | 228.5059 | 0.240689 | 48.2769 |
| 5% NiOZSM-5 | 206.8064 | 0.252456 | 51.4085 |
| 5% ZnOZSM-5 | 207.5806 | 0.256595 | 50.9644 |
| 7% FeOZSM-5 | 294.3253 | 0.279381 | 42.6424 |
| 7% NiOZSM-5 | 282.7488 | 0.27861 | 44.3677 |
| 7% ZnOZSM-5 | 255.8556 | 0.26559 | 46.3254 |
| 10% FeOZSM-5 | 284.2756 | 0.260572 | 41.9182 |
| 10% ZnOZSM-5 | 228.4006 | 0.262092 | 48.1762 |
| 10% NiOZSM-5 | 250.0925 | 0.252126 | 44.2589 |
| 20XII31KHMD(Ni-1) | 68.9364 | 0.132902 | 57.019 |
| 20XII25JHMD(Fe-1) | 197.0129 | 0.040275 | 21.4268 |
| 20XII31KHMD(Zn-1) | 84.4293 | 0.080983 | 42.4431 |
| 20XII03JHMD(Nl-2) | 109.2365 | 0.241019 | 76.7563 |
| 20XII03JHMD(Fe2) | 29.3045 | 0.011405 | 24.8016 |

Table 3.1 shows the BET surface area, pore volume and sizes of the zeolite systems synthesized by IWI and co-crystallisation. For the samples promoted by IWI, all the surface areas were more than 200 m²/g, the largest surface area was exhibited by the catalyst promoted with 3% ZnO. On the average, the samples synthesized with 5% MO loadings exhibited the least surface areas. Though the surface areas were all above 200 m²/g, generally the promotion by IWI reduced the surface area of the parent zeolite (308 m²/g) This could possibly be a s a result of the MO reducing the catalyst total coverage area.

It can be seen that most of the co crystallized samples have low surface areas which we connect to the low crystallization times of 24 hours. However, most of the samples are mesoporous with a mean pore size of 4.5 nm. The low crystallization times of 24 hours could have resulted in the formation of zeolite systems with low surface areas and we also do not exclude the none participation of some of the MO as a contributing factor to the low surface area.

It shall be understood that the examples are provided for illustrating the invention further and to assist a person skilled in the art with understanding the invention and are not meant to be construed as unduly limiting the reasonable scope of the invention.

1. A method for making an oligomerisation catalyst, which method comprises the steps of:
mixing alumina, silica, metal oxide, a template source; and
effecting the deactivation of the Bronsted sites by attachment of the metal thereto.

2. A method for making an oligomerisation catalyst as claimed in claim 1, wherein aluminosilicate starting gel is prepared from silica gel, aluminum nitrate nona hydrate and aluminium sulphate, alkyl salts are used as templating agents and one of nitrate of zinc, iron and nickel (M) were added to the mixture for co-crystallisation.

3. A method for making an oligomerisation catalyst as claimed in claim 2, wherein the SiO₂/MO ratio is selected between 100 and 10.

4. A method for making an oligomerisation catalyst as claimed in claim 3, wherein co-crystallisation is carried out in a hydrothermal synthesis reactor.

5. A method for making an oligomerisation catalyst as claimed in claim 4, wherein the hydrothermal synthesis is carried out for about 24 hours at a temperature of about 190° C., and after crystallisation the reactor is allowed to cool followed by filtration with distilled water until the filtrate had the same pH as the distilled water, and the obtained crystals are then dried and calcined, and the crystals were then protonated by stirring with 1 M ammonium chloride at 90° C.

6. A method for making an oligomerisation catalyst as claimed in claim 1, which includes the step of incipient wetness impregnation, wherein a protonised form of zeolites is obtained by calcining NH₄-ZSM-5 (zeolyst pellets) in a stream of Nitrogen at about 500° C. for about 3 hours, and nitrates of zinc, iron and nickel, M, were dissolved in water is then used to achieve a loading of 1 to 10% via incipient wetness, and the impregnated catalyst is then dried and calcined.

7. A method for making an oligomerisation catalyst as claimed in claim 1, which includes mechanical mixing, the protonised form of NH₄-ZSM-5 is mechanically mixed with nitrates of M, M being selected from zinc, iron and nickel, and the mixture is then calcined with MO % being 1, 0.2 and 0.04.

8. A method for oligomerising olefins, which method include the step of contacting olefins with the catalysts manufactured according to the method as claimed in claim 1, under oligomerisation reaction conditions.

9. A catalyst manufactured by the method as claimed in claim 1.

10. (canceled)

11. (canceled)

12. (canceled)

13. A method for oligomerising olefins, which method include the step of contacting olefins with the catalysts manufactured according to the method as claimed in claim 2, under oligomerisation reaction conditions.

14. A method for oligomerising olefins, which method include the step of contacting olefins with the catalysts manufactured according to the method as claimed in claim 3, under oligomerisation reaction conditions.

15. A method for oligomerising olefins, which method include the step of contacting olefins with the catalysts manufactured according to the method as claimed in claim 3, under oligomerisation reaction conditions.

16. A method for oligomerising olefins, which method include the step of contacting olefins with the catalysts manufactured according to the method as claimed in claim 4, under oligomerisation reaction conditions.

17. A catalyst manufactured by the method as claimed in claim 2.

18. A catalyst manufactured by the method as claimed in claim 3.

19. A catalyst manufactured by the method as claimed in claim 4.

20. A catalyst manufactured by the method as claimed in claim 5.

21. A method for oligomerising olefins, which method include the step of contacting olefins with the catalysts manufactured according to the method as claimed in claim 5, under oligomerisation reaction conditions.

22. A method for oligomerising olefins, which method include the step of contacting olefins with the catalysts manufactured according to the method as claimed in claim 6, under oligomerisation reaction conditions.

23. A catalyst manufactured by the method as claimed in claim 6.
